(19) United States
(12) Patent Application Publication
(10) Pub. No.: US 2018/0306785 A1
(43) Pub. Date: Oct. 25, 2018

(54) LATERAL FLOW ASSAY DEVICES AND METHODS

(71) Applicant: Wellbeing Technologies Corp., Princeton, NJ (US)

(72) Inventor: Joseph Z. Huang, Plainsboro, NJ (US)

(73) Assignee: Wellbeing Technologies Corp., Princeton, NJ (US)

(21) Appl. No.: 15/770,886

(22) PCT Filed: Oct. 26, 2016
(86) PCT No.: PCT/US16/58859

§ 371 Date: Apr. 25, 2018

Related U.S. Application Data
(60) Provisional application No. 62/246,588, filed on Oct. 26, 2015.

(51) Int. Cl. G01N 33/558; G01N 33/543; B01L 3/00

(52) U.S. Cl. CPC G01N 33/558; G01N 33/54386; B01L 3/5029; B01L 2300/0825; B01L 3/5023

(57) ABSTRACT

This application discloses a lateral flow assay method and device for quick collection and testing of chemical or biochemical samples by immunoassay or Immunochromatographic assays and use of the devices or methods for diagnosing diseases or conditions in a subject. The device includes a fluid sample collector and a test strip for collecting fluid samples and detecting biological analytes in it semi-quantitatively or quantitatively.

### CROSS REFERENCE TO RELATED APPLICATIONS

This application claims priority to U.S. Provisional Application No. 62/246,588, filed Oct. 26, 2015. The content of the application is incorporated herein by reference in its entirety.

### FIELD OF THE INVENTION

This invention relates to a lateral flow assay method and device for quick collection and testing of chemical or biochemical samples by immunoassay or Immunochromatographic assays.

### BACKGROUND OF THE INVENTION

Lateral flow assay or simple strip tests based on immunoassay or Immunochromatographic assays have been in existence for several decades, principally based on use of a series of capillary membranes, such as pieces of porous paper or sintered polymer sheets, for collecting and testing samples.

The benefits of lateral flow tests include: user-friendly format, very short time to obtain test results, long-term stability over a wide range of climates, and relatively low cost to make. These features make lateral flow tests ideal for applications such as home testing, rapid point-of-care testing, and testing in the field for various environmental and agricultural analytes.

Currently, two main methods are used for application of a sample to the lateral flow test. The first one involves direct application of a sample to the sample membrane through an opening above the sample membrane on the plastic housing. In the second method, the sample membrane is extended out of the plastic housing through a small opening at its end. A fluid sample is collected in a small container. By merging the extended sample membrane into the sample liquid, the sample is drawn into the sample membrane through the capillary force.

Both of these two sample collection methods have some drawbacks. For the first method, the amount of the sample that is applied to the sample membrane is hardly controllable, and thus a precise pipette is usually needed to apply a certain amount of sample onto the sample membrane. For the second method, the amount of sample that is collected depends on 1) the time that the extended sample membrane is inserted into the fluid sample, and 2) the depth that the extended sample membrane is merged into the sample liquid level; therefore, the amount of the collected sample is not accurate.

Although some recent publications have disclosed some designs and new developments of sample collections, see, e.g., B. O'Farrell, Topics in Companion An Med, 2016, which is incorporated by reference in its entirety, they are complex either in structure or in operation, thus resulting high costs.

### SUMMARY OF THE INVENTION

The present invention provides novel devices and methods for sample collection and lateral flow assay that can overcome the above-mentioned drawbacks and increase the efficiency and accuracy of the lateral flow assay.

In one embodiment the present invention provides a lateral flow assay device comprising a test strip and a sample collector. The test strip comprises a top housing, a bottom housing, and a series of capillary membranes, including a sample membrane, a conjugate membrane, an assay membrane, and an absorbent membrane. The top housing and the bottom housing provide a chamber that accommodates the series of capillary membranes in the position inside.

In some embodiments, the top housing comprises an opening that provides a window to detect results of reactions on the assay membrane. In some embodiments, the recesses on the both sides of the housing provide a position for printing or attaching a Bar or QR code and/or a position for the detection window. The bar or QR code can be on either the top housing or the bottom housing. In some embodiments, a well as an upward extended portion of the top housing accommodates a pad of sample collector.

In some embodiments the front of the top housing comprises a tilting openable lid that allows the sample collector to be covered at its close position, and allows the sample collector to slide out at its open position. In some embodiments, one or more convex structures are inside the lid, which presses the sample collecting pad down to touch the sample pad on the bottom housing so that the aqueous sample soaked on the sample pad is transferred to the sample membrane.

In another embodiment at least one sliding door underneath the detection window and on the top of the assay membrane protects the assay membrane from moistures and contaminations during sample collection. In one embodiment, the sliding door can be slid to the other side manually or by a mechanism in the reader before the detection, which allows the results on the assay membrane to be read.

The lateral flow assay methods and devices described herein can be used to accurately collect a certain amount of sample without needing to use expensive laboratory pipettes. The sample collector can be used to draw certain amount of any fluid sample and transfer it to the sample membrane in the lateral flow device. The device is suitable for carrying out the lateral flow assay in quantitative or semi-quantitative measurement. Thus, in another aspect, the present invention also provides diagnostic methods for diseases or conditions in a subject using the lateral flow assay devices or methods.

These advantages and other aspects of the present invention may be better appreciated through the following drawing, detailed descriptions, and claims.

### BRIEF DESCRIPTION OF THE DRAWINGS

FIG. 1 illustrates an explanatory view that shows an embodiment of a lateral flow assay device that consists of a test strip and a sample collector.

FIG. 2 illustrates a top view of the test strip in FIG. 1.

FIG. 3 illustrates a sectional view of the test strip in FIG. 1.

FIG. 4 illustrates an explanatory view that shows the sample collector in FIG. 1.

FIG. 5 illustrates an explanatory view of another embodiment of lateral flow assay device that consists of two test strip and a sample collector.

FIG. 6 illustrates an explanatory view that shows the embodiment given in FIG. 5.

FIG. 7 illustrates a sectional view of the embodiment shown in FIG. 6.

FIG. 8 illustrates another explanatory view of the embodiment illustrated in FIG. 5.

FIG. 9 and FIG. 10 are explanatory views of the embodiment given in FIG. 5.

### DETAILED DESCRIPTION OF THE INVENTION

The present application discloses lateral flow assay devices and methods for collecting and analyzing chemical or biological samples and uses of these devices or methods in diagnosing diseases or conditions in a subject.

In one aspect the application discloses a device of a lateral flow assay for quick collecting and testing chemical or biochemical samples by immunoassay or Immunochromatographic assays, comprising:

1) a test strip with a bottom housing, a top housing, and a series of capillary membranes; and

2) one or more sample collectors, each with a sample collecting pad.

In one embodiment, a bar or QR code is printed on or attached to either the top or the bottom housing of the device, which gives the test strip a unique code for use.

In another embodiment, the series of capillary membranes comprise a sample membrane, a conjugate membrane, an assay membrane, and an absorbent membrane.

In another embodiment, the device comprises one, two or three series of the capillary membranes, each series comprising a sample membrane, a conjugate membrane, an assay membrane, and an absorbent membrane.

In another embodiment, an extended well is on the top housing to accommodate the sample collecting pad of the sample collector.

In another embodiment, on the top housing are installed one or more extended wells to accommodate two or more sample collecting pads for collecting multiple samples.

In another embodiment, at the bottom of the extended chambers is installed a filter pad that filters and transfers the sample solution from the sample collector to the sample membrane.

In another embodiment, the sample collecting pad on the sample collector is a membrane made from cotton, sponge, glass fiber, or other materials.

In another aspect, the application discloses a lateral flow assay device for collecting and testing chemical or biochemical samples by immunoassay or immunochromatographic assays, comprising:

1) a test strip with a bottom enclosure, a top housing, a lid, and a series of capillary membranes; and

2) at least one sample collector comprising a sample collecting pad;

3) at least one detection window with a sliding door.

In one embodiment, the series of capillary membranes consist of a sample membrane, a conjugate membrane, an assay membrane, and an absorbent membrane.

In another embodiment, the device comprises two or more series of capillary membranes, each comprising a sample membrane, a conjugate membrane, an assay membrane, and an absorbent membrane.

In another embodiment, at the front of the top housing is installed a tilting-openable lid that allows the sample collector to be covered at its close position, and allows the sample collector to slide out at its open position.

In another embodiment, one or more convex structures are installed on the inside of the lid, which presses the sample collecting pad down to contact the sample pad on the bottom housing.

In another embodiment, the sample collecting pad on the sample collector is a membrane made from cotton, sponge, glass fiber, or other materials.

In another embodiment, at least one sliding door is installed underneath the detection window and on the top of the assay membrane

In another aspect, the present application discloses a method of collecting and assaying a biological sample from a subject comprising use of a device according to any embodiment disclosed herein.

In one embodiment, the biological sample can be an in vitro sample pre-collected from the subject.

In another embodiment, the biological sample can be an in vivo sample collected directly from the body of the subject using the device.

In another embodiment, the subject is a mammalian animal.

In another embodiment, the subject is a human.

In another aspect, the present application discloses a diagnostic method for determining a disease or condition in a subject, comprising use of a lateral flow assay device according to any embodiment disclosed herein in collecting and/or assaying a biological sample from the subject.

### Definitions

The term “membrane” as used in the following refers to a sheet made from primarily hydrophobic or hydrophilic materials, such as nitrocellulose, cellulose acetate, or glass fiber. The membrane provides the capillary force to transport chemical or biological fluid from a sample membrane, through a conjugate membrane, an assay membrane, to an absorbent membrane.

The term “microspheres” means a dried form of bio-active particles, such as latex and nanoparticles of gold, fluorescent or magnetic labeled particles, that retain an element, a compound, and/or a molecule in a liquid state, in a solid state, or in a semi-solid gel state. There are several sizes and polymers to choose from. Usually the microspheres conjugated with antibodies or antigens migrate down the membrane upon introduction of the sample.

A biological sample is any fluid sample existing in a subject that can be conveniently collected directly by a sample collector, in particular the sample collecting pad, as disclosed herein, including but not limited to blood, saliva, urine, tears, vagina fluid, etc.

The term “subject”, as used herein, refers to a mammalian animal, preferably a human.

Each of these membranes provides the capillary force to transport chemical or biological fluid from a sample membrane, through a conjugate membrane, an assay membrane, to an absorbent membrane. The sample membrane acts as a sponge and holds a sample fluid. Once soaked, the fluid migrates to the conjugate membrane with microspheres, a dried format of bio-active particles, such as latex and nanoparticles of gold, fluorescent or magnetic labeled particles. While the sample fluid dissolves the dried bio-active particles in the conjugate membrane and flows through the porous structure, chemical reactions take place between the target molecules (e.g., an antigen) in the sample and their reactive partners (e.g., antibody) immobilized on the surface of the particles in the conjugate membrane. In this system, the analytes bind to the particles while migrating further through the assay membrane. On the assay membrane, there is one or more area where other molecules may be immobilized. As the sample-conjugate fluid reaches these areas, the analytes may be bound or not bound to the immobilized molecules, depending on the specificities of the analyses and the immobilized molecules. Typically there are at least two areas: one (the control) that captures any particle and thereby shows that reaction conditions and analysis are fine, the other containing a specific capture molecule which only captures those particles onto which the analyte molecules have been immobilized. After passing these reaction areas the fluid enters the final membrane, the absorbance, which simply acts as a waste container. Lateral Flow Tests can operate as either competitive or sandwich assays.

The analytes that are bound or not bound to the immobilized molecules in the assay membrane can be visually detected qualitatively or semi-quantitatively. Some of the more common lateral flow tests currently on the market are tests for pregnancy, Strep throat, and Chlamydia. These are examples of conditions for which a quantitative assay is not necessary.

The series of capillary membranes that are often very fragile are placed on a backing to enhance strength. A plastic housing is the case of lateral flow test strip. A foil pouch is used to seal the test strip in. Desiccant can be added into the pouch separately or incorporated into the absorbent membrane, which is used to keep ingredients dry during storage before use.

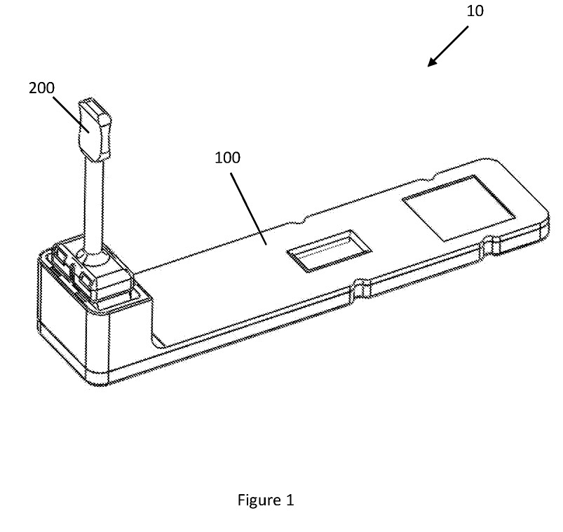

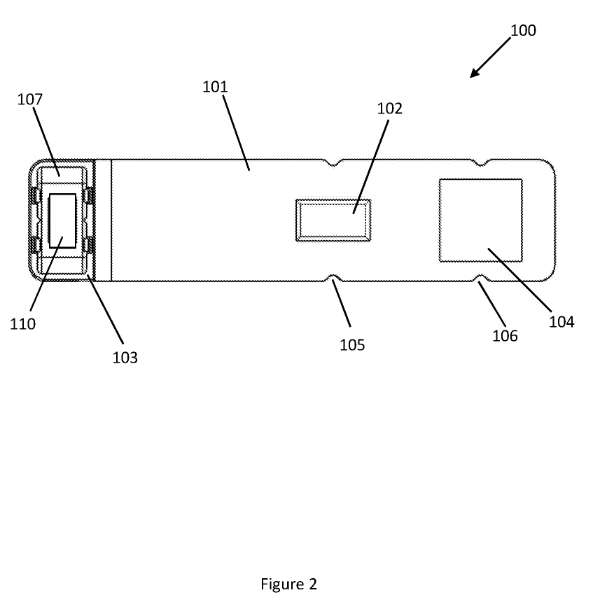

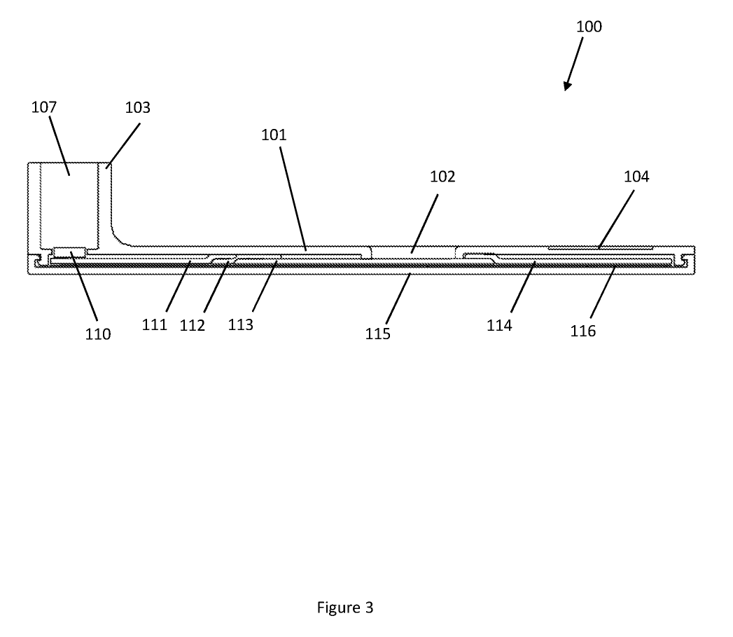

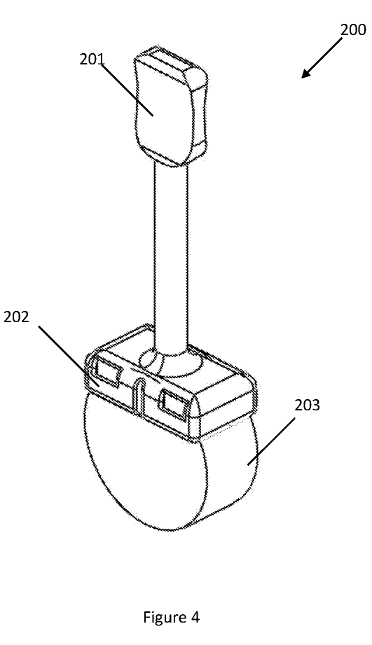

FIG. 1 illustrates an embodiment of the lateral flow device disclosed herein. Referring to FIG. 1, the lateral flow device 10 consists of a test strip 100 and a sample collector 200. FIG. 2 and FIG. 3 show the detailed components of the test strip 100, and FIG. 4 the details of the sample collector.

The test strip 100 comprises a top housing 101, a bottom housing 115, and a series of capillary membranes. The series of capillary membranes include a sample membrane 111, a conjugate membrane 112, an assay membrane 113, and an absorbent membrane 114. The top housing 101 and the bottom housing 115 provide a chamber that accommodates the series of the capillary membranes in the position inside. On the top housing 101, there is an opening 102 that provides a window to detect results of reactions on the assay membrane 113, a recess 104 that defines a position for a bar or QR code, and a well 107 formed by an above extended housing 103 that accommodates the pad 203 of the sample collector 200. On the each side wall of the top housing 101 and the bottom housing 115, there are two recesses 105 and 106, which define the positions of the opening 102 and the recess 104 on the top side of the housing 101. The recesses on the both side walls allow a sliding tack switch to give signals of the positions of the opening 102 and the recess 104 when the test strip 100 is inserted into a reader for the test results. When the signal from the sliding tack switch is given, a CCD camera in the reader can be triggered to take a picture of the bar or QR code at the position of the recess 106, a picture of the test result at the position of the opening 105. On the both top and the bottom housing 101 and 115, there are mechanisms that lock the top and the bottom housing together.

The extended well 107 accommodates the pad 203 of the sample collector 200. On the bottom of the cell 107 there is a filter pad 110 that transfers the sample solution from the sample collector 200 to the sample membrane 111, and at the same time blocks any particles and/or food residues in the sample solution.

The sample membrane 111 is overlapped on the conjugate membrane 112, the conjugate membrane 112 on the assay membrane 113, and the absorbent membrane 114 on the assay pad 113, too. With this configuration, the sample solution is drawn from the sample collector 200, to the filter pad 110, and then to the sample membrane 111. By capillary force of the porous membranes, the sample solution is continuously drawn from the sample membrane 111 to the conjugate membrane 112, the sassy membrane 113, and then the absorbent membrane 114.

The sample collector 200 as shown in FIG. 4 comprises a handler 201, a round pad 203, and a pad holder 202. When soaked into a liquid when the target analytes are present, the pad 203 collects the sample. The pad 203 can be made from cotton or cellulose with any suitable size, for example, with a diameter of 5-10 mm and thickness 2-3 mm. As the size of the pad 203 is certain, the certain volume saturated into the collecting pad 203 can be accurate, up to 0.1 to 0.25 milliliter, depending on its diameter and thickness.

For example, to use the present device for saliva sample testing, the pad 203 of the sample collector 200 can be inserted directly into a subject's mouth for about 60 seconds to collect the saliva sample. Then the cotton/cellulose pad 203 of the sample collector 200 is placed into the well 107 on the test strip 100. The saliva sample is drawn from the pad 203 to the sample membrane 111 through the filter pad 110 on the bottom of the well 107. By the capillary force of the membranes, the saliva sample is transported from the sample membrane 111, through the conjugate membrane 112 and the assay membrane 113, to the absorbent membrane 114.

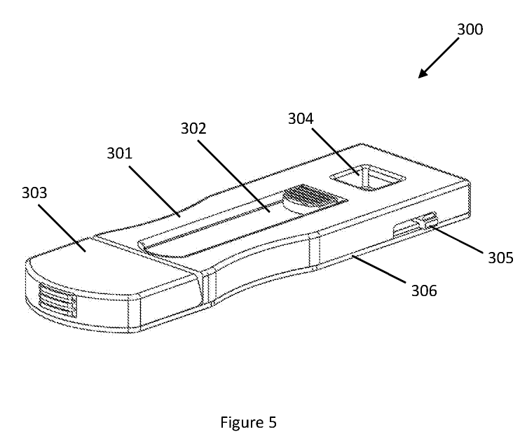

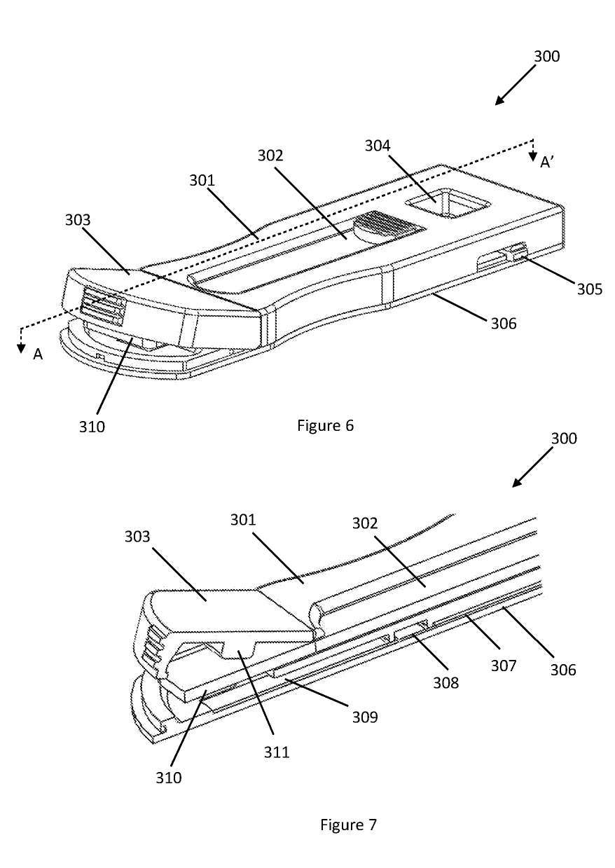

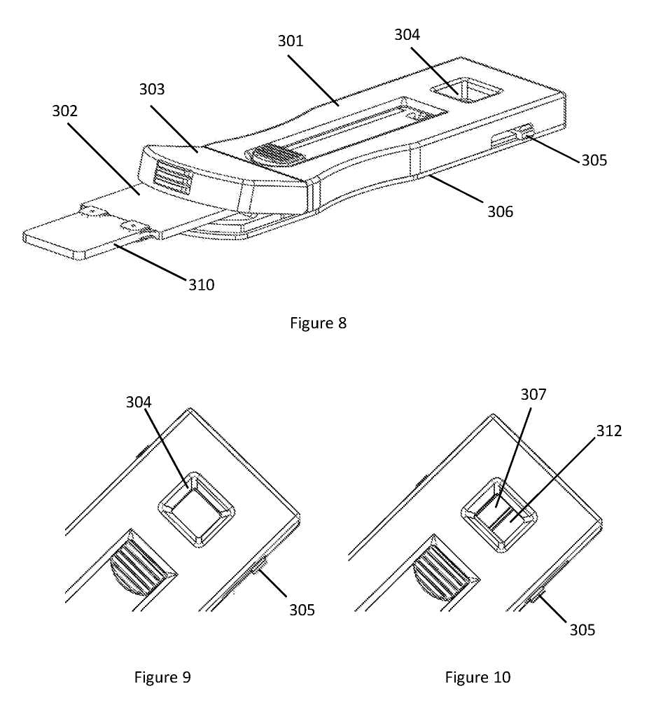

FIG. 5 illustrates another embodiment of the lateral flow device comprising an integrated sample collector. The lateral flow device 300 comprises a bottom housing 306 for two test strips, a top enclosure 301, a tilting-openable lid 303, a sliding door 305 for the detection window 304, and a sample collector 302. FIG. 6, FIG. 7, and FIG. 8 show the detailed structures of the test strip, the sample collector 302 and its use. FIG. 9 and FIG. 10 provide the details of the detection window 304 and the sliding door 305.

FIG. 6 shows the lateral flow device 300 with the tilting-openable lid 303 open. FIG. 7 shows the sectional structure from the section A-A′ in FIG. 6. One of the test strips consists of a sample membrane 309, a conjugate membrane 308, an assay membrane 307, and an absorbent membrane (not shown in the figures) that are placed on the bottom housing 306. The sample collector 302 consists of a sample collecting pad 310 at its front. The sample collector 302 can be slid out from the opening of the lid 303, as shown in FIG. 8. After the sample collecting pad 310 is saturated with a fluid sample, the sample collector 302 can be slid back to the housing of the lid 303. Upon closing of the lid 303, a convex structure 311 on the lid 303 is pressed on the soaked sample collecting pad 310 so that the fluid sample is transferred from the sample collecting pad 310 to the sample membrane 309. The sample collecting pad 310 can be made from cotton or cellulose with its size from 8×8 mm to 12×12 mm, and thickness 1-3 mm.

FIG. 9 illustrates that the detection window 304 is closed by the sliding door 305, which can prevent the assay membranes 307 and 312 from moisture and contamination during the sample collection. Before the detection, the sliding door 305 can be slid to the other side by hand or by mechanism in a reader, as shown in FIG. 10, that allows the results on the assay membranes 307 and 312 to be read.

Advantages of the methods and devices as disclosed herein over the existing lateral flow devices include, but are not limited to:

1) that the sample is able to be collected conveniently and accurately;

2) that no expensive liquid handling equipment such as pipette is needed to apply the sample onto the test strip; and

3) that the volume of the sample applied to the test strip is controlled and the measurement is quantitative or semi-quantitative.

Although the invention herein has been described with reference to the particular embodiments, it is to be understood that these embodiments are merely illustrative of certain principles and applications of the present invention. Numerous modifications may be made to the illustrative embodiments and other arrangements may be devised without departing from the spirit and scope of the present invention as defined by the following claims.

1. A device of a lateral flow assay for quick test chemical or biochemical samples by immunoassay or Immunochromatographic assays, comprising:
1) a test strip with a top housing, a bottom housing, and a series of capillary membranes; and
2) one or more sample collectors, each with a sample collecting pad.

2. The device of claim 1, wherein a bar or QR code is printed on or attached to either the top or the bottom housing, which gives the test strip a unique code for use.

3. The device of claim 1, wherein the series of capillary membranes comprise a sample membrane, a conjugate membrane, an assay membrane, and an absorbent membrane.

4. The device of claim 1, wherein the device comprises one, two, or three series of the capillary membranes, each series comprising a sample membrane, a conjugate membrane, an assay membrane, and an absorbent membrane.

5. The device of claim 1, wherein an extended well is on the top housing to accommodate the sample collecting pad of the sample collector.

6. The device of claim 1, wherein on the top housing are installed two or more extended wells to accommodate two or more sample collecting pads for collecting multiple samples.

7. The device of claim 1, wherein at the bottom of the extended chambers is installed a filter pad that filters and transfers the sample solution from the sample collector to the sample membrane.

8. The device of claim 1, wherein the sample collecting pad on the sample collector is a membrane made from cotton, sponge, glass fiber, cellulose, or other materials.

9. The device of claim 1, wherein the sample collecting pad on the sample collector is a membrane with a diameter at 5-10 mm and a thickness 2-3 mm.

10. A lateral flow assay device for collecting and testing chemical or biochemical samples by immunoassay or Immunochromatographic assays, comprising:
1) a test strip with a top enclosure, a bottom housing, a lid, and a series of capillary membranes; and
2) at least one sample collector comprising a sample collecting pad;
3) at least one detection window with a sliding door.

11. The device of claim 10, wherein the series of capillary membranes consist essentially of a sample membrane, a conjugate membrane, an assay membrane, and an absorbent membrane.

12. The device of claim 10, comprising two or more series of capillary membranes, each comprising a sample membrane, a conjugate membrane, an assay membrane, and an absorbent membrane.

13. The device of claim 10, wherein at the front of the top housing is installed a tilting-openable lid that allows the sample collector to be covered at its close position, and allows the sample collector to slide out at its open position.

14. The device of claim 10, wherein one or more convex structures are installed on the inside of the lid, which press the sample collecting pad down to contact the sample pad on the bottom housing.

15. The device of claim 10, wherein the sample collecting pad on the sample collector is a membrane made from cotton, sponge, glass fiber, cellulose, or other materials.

16. The device of claim 10, wherein at least one sliding door is installed underneath the detection window and on the top of the assay membrane.

17. A method of collecting and assaying a biological sample from a subject comprising use of a device of claim 1.

18. The method of claim 17, wherein the biological sample is an in vitro sample pre-collected from the subject.

19. The method of claim 17, wherein the biological sample is an in vivo sample collected from the subject using the device.

20. The method of claim 17, wherein the subject is a mammalian animal.

21. (canceled)
